(19) United States
(12) Patent Application Publication
(10) Pub. No.: US 2007/0166321 A1
(43) Pub. Date: Jul. 19, 2007

(54) Compositions and Methods for Reducing Cholesterol and Inflammation

(75) Inventor: Bryant Richard Villeponteau, San Diego, CA (US)

Correspondence Address: BRYANT R. VILLEPONTEAU, 4953 SMITH CANYON CT., SAN DIEGO, CA, 92130, US

(73) Assignee: Bryant Richard Villeponteau, San Diego, CA (US)

(21) Appl. No.: 11/460,961

(22) Filed: Jul. 28, 2006

Related U.S. Application Data
(60) Provisional application No. 60/766,372, filed on Jan. 13, 2006.

(51) Int. Cl. A61K 36/06; A61K 31/455; A61K 31/401; A61K 31/366; A61K 31/22; A61K 31/385

(52) U.S. Cl. 424/195.16; 514/356; 514/423; 514/440; 514/460; 514/548; 514/724

(57) ABSTRACT

Statin therapy has revolutionized the treatment of cardiovascular disease, but not all patients can take the appropriate level of statins because of their side effects. The present invention provides compositions that provide potent cholesterol lowering while minimizing the damaging side effects to liver, muscles, and neurons, and has the added benefit of reducing chronic systemic inflammation, which is an independent determinant of cardiovascular disease and all-cause mortality. The current invention presents pharmaceutical compositions for reducing cholesterol and chronic systemic inflammation comprising therapeutically effective amounts of: at least one lipid-lowering agent chosen from HMG-CoA reductase inhibitors, high-dose controlled-release niacin, red yeast rice, or policosanol; and at least one antiinflammatory natural product chosen from alpha-lipoic acid and corosolic acid. To those in need of such treatment, the current invention also provides safe methods for reducing high serum cholesterol or chronic inflammation, or for simultaneously reducing both cholesterol and chronic inflammation via treatment with therapeutically effective daily doses of pharmaceutical compositions as described herein. The present invention provides mammals with compositions and methods for concurrently reducing cholesterol and inflammation as a prevention or treatment for many age-related diseases and disorders.

### FIELD OF THE INVENTION

The present invention relates to compositions and methods for safely reducing serum levels of total cholesterol, low-density lipoproteins (LDL), oxidized LDL, and triglycerides (TG), whereby lipid-lowering agents such as HMG-CoA reductase inhibitors, high-dose controlled-release niacin, red yeast rice, or policosanol are combined with alpha-lipoic acid to reduce the liver, muscle, and neuronal damage often associated with using said lipid-lowering agents. The present invention also relates to the reduction of chronic systemic inflammation, which is an independent indicator of cardiovascular disease, many other chronic diseases, and all-cause mortality. The metrics for measuring changes in systemic inflammation consists of reducing serum levels of high-sensitivity C-reactive protein (CRP), Interleukin 6 (IL-6), and/or tumor necrosis factor alpha (TNF-alpha). To reduce inflammation along with high cholesterol levels, the present invention adds the natural antiinflammatory agent corosolic acid to HMG-CoA reductase inhibitors and alpha-lipoic acid. Given that drug compliance is always a problem with long term drug use in healthy individuals, the invention presents combinations that can be taken as a single once-a-day capsule, softgel, or tablet that simultaneously treat both high cholesterol and high serum indices of inflammation (CRP, IL-6, and TNF-alpha levels). When the said compositions are taken in therapeutically effective doses, the current invention also provides methods for the prophylaxis and treatment of age-related disorders such as atherosclerosis, heart disease, stroke, neurodegenerative diseases, and elevated plasma levels of LDL, TG, CRP, IL-6, and/or TNF-alpha for mammals in need of such treatment.

### BACKGROUND OF THE INVENTION AND DESCRIPTION OF THE PRIOR ART

Atherosclerosis (or arteriosclerosis) is the term used to describe the slow and progressive narrowing and hardening of the arteries, which typically starts in childhood. Although usually asymptomatic until age 35 to 60, atherosclerosis typically leads to aneurysms, thrombosis, ischemia, embolism formation, or other vascular insufficiencies. The disease process can occur in any artery in the human body and with the typical western diet progresses systemically as a function of age throughout most arterial vessels in the body. Thus, by their late 50s, the average westerner is already endangered by moderate to severe forms of atherosclerosis, which is invariably worse if the person also smokes. This widespread atherosclerosis directly promotes the progression of most of the age-related diseases and the increased mortality with age.

Atherosclerosis and the subsequent cardiovascular disease are typically the biggest killers in western societies. For example, atherosclerosis in the carotid or intracerebral arteries that supply the brain can result in ischemic stroke. Cardiac insufficiency or heart attack occurs when the coronary arteries that supply oxygen and nutrients to the heart myocardium are blocked, and extremity pain and/or gangrene occur when peripheral arteries to an extremity become affected. Death finally occurs when the blood supply to an essential organ is sufficiently compromised, but debilitating morbidity can be present long before death, as often occurs in stroke patients and heart attack survivors.

While the detailed mechanisms of atherosclerosis are not completely understood, much is now known about the complex causes of the atherosclerosis process. Atherosclerosis apparently begins when the intima layer of the artery, the inner lumen endothelium, becomes damaged by oxidized low-density lipoprotein (LDL) cholesterol, infectious agents, hyperglycemia, homocystine, and/or oxidative toxins. Oxidized LDL cholesterol begins accumulating in the damaged vascular endothelial cells, leading to fatty streaks, vascular inflammation, and smooth muscle cell proliferation. The accumulating oxidized LDL in the fatty streaks activates inflammatory processes by up-regulation nuclear factor kappa-B, expression of numerous cytokines (especially IL-6 and TNF-alpha), and the recruitment of monocytes/macrophages that are injurious to the endothelium. Circulating monocytes then infiltrate the intima of the vessel wall, and these tissue macrophages act as scavenger cells, taking up more oxidized LDL cholesterol and forming the characteristic foam cells of early intima plaques.

The endothelia intima of the artery becomes markedly thickened and inflamed by the accumulating foam cells engorged with oxidized LDL and by external fibrin material. Hardening of the arterial wall is due to assorted depositions within the plaque including lipids, oxidized cholesterol crystals, and calcium salts. These deposits restructure the arteries into rigid tubes. The thickening and hardening of the intima diminishes blood flow (ischemia) to the brain, heart, and/or the extremities. Moreover, the enlarged plaque damage causes blood platelets to adhere, allowing blood clots to form on the plaques, which can lead to full occlusion (stenosis) of the vessel and hypoxia in tissues totally cut off from normal blood circulation.

From the above description of the importance of cholesterol in the overall etiology of atherosclerosis, it is not surprising that lowering cholesterol levels is widely prescribed for the prevention and treatment of cardiovascular disease. Indeed, elevated serum cholesterol (>200 mg/dL) is a known risk factor for cardiovascular disease. Experts typically recommended that individuals at high risk for cardiovascular disease decrease serum cholesterol levels through dietary changes, a program of physical exercise, and other lifestyle changes. However, even where saturated fat and cholesterol are severely restricted in the diet, the liver remains able to synthesize large quantities of cholesterol for necessary bodily functions and often overproduces what is optimal for cardiovascular health. Therefore, drug intervention to block cholesterol production in the liver is the preferred treatment for those individuals (perhaps a majority of the population) unable to reduce total cholesterol below acceptable levels by dietary or lifestyle changes.

The liver is the primary organ responsible for cholesterol homeostasis and is the main determinant of plasma cholesterol levels. The liver is the site of synthesis and secretion of very low density lipoproteins (VLDL), which are subsequently metabolized to low density lipoproteins (LDL) in the circulation. LDL comprises the predominant cholesterol-carrying lipoproteins in the plasma and high LDL levels are readily taken up by atherosclerotic plaques. This is in marked contrast to the high-density lipoproteins (HDL), which serve as ‘good’ cholesterol that can help clear cholesterol from serum and atherosclerotic plaques. Therefore, the best indicator of cardiovascular risk is the ratio of LDL to HDL with optimal ratios approaching 2 in order to minimize cardiovascular risk.

While several cholesterol-lowering agents were discovered during the fifties and sixties, most had undesirable side effects. In the early seventies, investigators found that the production of cholesterol by the liver could be controlled by inhibiting the enzyme 3-hydroxy-3-methylglutaryl-CoA reductase (HMG Co-A reductase), which regulates the conversion of HMG-CoA to mevalonic acid. The HMG-CoA reductase inhibitors are called statins and include atorvastatin, cerivastatin, fluvastatin, mevastatin, lovastatin, pravastatin, rosuvastatin, and simvastatin. The statins have been shown to be highly effective in reducing LDL cholesterol and atherosclerosis. Many statin drugs also raise good HDL cholesterol to some degree, which adds to their benefit in reducing atherosclerosis.

As a group, statin drugs are relatively safe, but do have both mild and some serious side effects [1]. Mild side effects that usually do not require medical attention include stomach upset or pain (e.g. acid indigestion, gas, diarrhea, nausea, or mild vomiting), headache, joint pain, and occasional tiredness. Other less common statin side effects are more serious: dark yellow or brown urine; difficulty in urination; fever; muscle pain, tenderness or cramps; redness, itching, or skin rash; unusual tiredness or weakness; and yellowing of the skin or eyes. Early warning of serious side effects of statin therapy is often revealed by liver enzyme assays. If the liver comes under inflammatory stress from statin drug use, certain liver enzymes will be elevated, which usually leads to a recommendation to stop statin use. Perhaps the most dangerous side effect is muscle inflammation, which can lead to mitochondrial leakage of oxidative free radicals. Up to 10% of statin users may have a mild form of muscle inflammation with a feeling of tiredness. Statin drugs can further promote instability in the mitochondria and the subsequent release of more oxidative free radicals. Overall, muscle inflammation and damage to mitochondria can cause muscle pain, cramps, and/or weakness. In more extreme cases, muscle mitochondria can disintegrate and release cytochrome 450 heme groups into the general circulation that can lead to iron-induced oxidative stress of the kidney and even kidney failure.

Long term use is the norm with statin therapy, but may present its own risks even if liver, muscle, or kidney damage does not appear during the first year of use. With long term use, there is a significant risk of developing myopathy [1, 2]. Long term statin use is also associated with higher risk for developing peripheral neuropathy [1-3], which appears to work via enhanced neuronal apoptosis [4]. Statin-induced myopathy and neuropathy are both serious risks for the millions of patients taking statins long term, which have not been adequately addressed by current treatment practices or by the prior art.

The current invention recognizes these side effects of statins and addresses a major cause of statin damage, which is damage to mitochondria and subsequent damage to muscle and neuronal cells via inflammation or apoptosis. The current invention uses the anti-inflammatory antioxidant alpha-lipoic acid to stabilize mitochondria and to protect against apoptosis in blood vessel endothelial, liver, muscle, and neuronal cells [5-7]. In animal work, alpha-lipoic acid has been reported to protect neurons from beta-amyloid, oxidative stress, and other neurotoxins [8-11]. In humans, alpha-lipoic has been shown to be an effective treatment for peripheral neuropathy and polyneuropathy in clinical trials [12-14]. While clinical trials on the effects of alpha-lipoic acid on myopathy have not yet been carried out, alpha-lipoic acid has been shown to have positive benefits for skeletal muscle cells in glucose and fatty acid uptake studies [15, 16]. All of these therapeutic effects of alpha-lipoic acid make it an ideal complement to statins to reduce the known statin side effects.

Cardiovascular patients who are treated with statins for 1 to 10 years have a very significant reduction in all-cause mortality of 39 to 46% compared to non-statin treated cardiovascular patients [17-19]. It is believed that part of this large reduction in all-cause mortality is independent of the lipid reducing effects of statins and is due to reduction of systemic inflammation by statins [20-22]. Strong evidence for the reduction of systemic inflammation by statins is given by the fact that statins substantially reduce biomarkers of inflammation such as CRP, IL-6, and TNF-alpha [23-26]. As recognized by the current invention, alpha-lipoic acid has also been shown to be antiinflammatory for bone and vascular endothelial cells and can suppress the pro-inflammatory serum levels of IL-1, IL-6, and TNF-alpha [27-30]. Therefore, statins and alpha-lipoic are synergistic antiinflammatory agents.

As an additional composition to reduce systemic inflammation, the current invention combines the natural antiinflammatory agent corosolic acid with alpha-lipoic acid and a statin. Corosolic acid is found in many plants native to Asia. Rich sources of corosolic acid are found in leaves of Lagerstroemia Speciosa L. or Perilla frutescens, which have traditionally been used in several Asian countries to treat diabetes. Like the diabetic drugs rosiglitazone (Avandia) and pioglitazone (Actos), corosolic acid apparently modulates peroxisome proliferator-activated receptors (PPARs) [31] to stimulate the cellular uptake of glucose. However, independent of its glucose lowering effect, corosolic acid also has potent antiinflammatory effects, as indicated by its ability to inhibit 12-O-tetradecanoylphorbol-13 acetate (TPA) induced inflammation with potency similar to the antiinflammatory drug indomethasin [32]. In more detailed TPA-induced inflammation studies, corosolic acid had a 50%-inflammation reduction index of ID50=0.03 mg, which compares well with ID50=0.3 mg for indomethacin and ID50=0.03 mg for hydrocortisone [33]. Moreover, unlike the glucose-lowering drugs rosiglitazone and pioglitazone, which also have independent antiinflammatory activity [34-36], corosolic acid does not significantly reduce glucose levels of non-diabetics [37] and thus may be safely used for its antiinflammatory properties in non-diabetics. Lagerstroemia Speciosa L. extracts standardized for corosolic acid (GlucoTrim) have potent glucose lowering activity [38], and have been sold for years as a nutritional supplement to maintain healthy glucose levels and to aid in weight loss. Based on its apparent safety and antiinflammatory potency, corosolic acid or plant extracts standardized for corosolic acid are excellent synergistic agents to combine with the systemic antiinflammatory efficacies of statins and alpha-lipoic acid.

There are no prior patents or published patent applications with a simple combination of statins and alpha-lipoic acid. The closest example to the current invention is the anti-cancer combination of statins with a selective COX-2 inhibitor and optional cystine and alpha-lipoic acid (US 20040092565, Kindness et al.). Likewise, there are no conflicting prior patents or published patent applications using combinations of statins with corosolic acid or alpha-lipoic acid with corosolic acid. On the use of corosolic acid, U.S. Pat. No. 6,716,459 (Matsuyama et al.), and patent application US 20050267055 (Matsuyama et al.) have claimed corosolic acid as a potential composition for improving glucose intolerance. Moreover, US patent application 20040072901 (Udell et al.) claims Lagerstroemia speciosa L extracts (standardized for corosolic acid) for the management of weight via glucose maintenance. Since the current invention uses statin compositions with corosolic acid (either purified corosolic acid or Lagerstroemia speciosa L extracts standardized for corosolic acid) to reduce inflammation irrespective of serum glucose levels or body weight, corosolic acid or its crude plant extracts are claimed by the current invention as a novel antiinflammatory agent that is synergistic with statins and alpha-lipoic acid.

Looking at non-statin substances that significant affect cholesterol levels, the plant derived long-chained aliphatic alcohol policosanol may be nearly as effective as the statins. Originally developed in Cuba from sugar cane, policosanol inhibits the biosynthesis of cholesterol in the liver by some mechanism that has yet to be elucidated. Policosanol is believed to be relatively effective in lowering LDL and raising HDL based on a number of small scale human clinical trials. However, policosanol has never undergone rigorous FDA trials and is therefore only available as an unapproved supplement of unknown reliability. Very few side effects have been reported so far, but that may reflect the small-scale nature of the clinical trials. It is likely that larger clinical trials of policosanol would reveal many of the same side effects observed with the statins. Therefore, alpha-lipoic acid and optional corosolic acid should be complementary to the cholesterol lowering effects of policosanol, in the same way as it is complementary to the statin drugs.

Other non-statin agents include red yeast rice and niacin. Red yeast rice contains a small amount of a statin-like compound, which could explain why it lowers cholesterol. However, the statin-like agent (lovastatin) in red yeast rice is found at very low levels compared to the pharmaceutically effective doses in statin drug therapy, so inhibition of HMG Co-A reductase may not be its major mechanism of action. This is also true of high-dose niacin, where the lipid-lowering mechanism is not well understood. Niacin must be taken at very high doses of 1 to 2 grams per day, which generates serious side effects in many patients. Moreover, some patients have actually seen their serum CRP level rise with high-dose niacin therapy. Combining these natural lipid lowering agents with alpha-lipoic acid and/or corosolic acid should further reduce cardiovascular risks by minimizing serum CRP levels and systemic inflammation.

In the prior art, most cholesterol-lowering combination therapies have presented a mix of lipid lowering agents described as having a synergistic hypolipidemic effect. For example, U.S. Pat. No. 6,933,291 (Qi et al.) and its predecessors claim compositions comprising (a) one or more phytosterols and/or phytostanols or a mixture thereof capable of reducing cholesterol absorption in the intestine, (b) a composition capable of inhibiting cholesterol biosynthesis, and (c) a composition capable of increasing cholesterol metabolism. In practice, cholesterol absorbing agents as specified in (a) are typically required at high dosage levels, which makes such agents more likely to have intestinal side effects in many patients. More generally, many combinations of existing lipid regulating agents are contraindicated or vary in effectiveness in different people, making it a complex exercise for physicians to proscribe the right combination for every patient requiring higher doses of LDL-reducing pharmaceuticals. As another example, U.S. Pat. No. 6,890,941 (Angres et al.) and its predecessors claim a composition comprising both a HMG-CoA reductase inhibitor and policosanol. This combination also suffers from greater complexity for those patients needing higher doses of the LDL-reducing compounds. Moreover, it is difficult to say what the benefits and side effects of this combo might be, as both HMG-CoA reductase inhibitor and policosanol act on liver biosynthesis and published clinical trials on the combination are not yet available.

In contrast to the above prior art, the combination therapy of the current invention typically has only one main cholesterol-lowering agent, so it is a simple mater to titrate the level of this one agent for patients needing greater cholesterol reduction. Alpha-lipoic acid and/or corosolic acid would typically stay at nearly the same levels for all patients to counteract the likely side effects of the cholesterol-lowering drug and to reduce systemic inflammation. As important, the two said natural products (alpha-lipoic acid and corosolic acid) have very low toxicity themselves at the recommended doses and thus should not complicate the combination theory with their own side effects.

Independent of serum cholesterol levels, chronic low-level inflammation is now believed to be a significant cause of many age-related disorders including Alzheimer's disease, cancer, cardiovascular disease, diabetes, and frailty in the elderly [39-43]. Atherosclerosis may be a major driver of other age-related disorders, as damaged vascular endothelium activates inflammatory processes by up-regulation cytokines like Interleukin 6 (IL-6) and C-reactive protein (CRP). Chronic systemic inflammation then disturbs many other organ systems leading to other age-related disorders. The antiinflammatory effects of the current invention containing a statin, alpha-lipoic acid, and corosolic acid will counteract the inflammatory dysfunctions linked to most of the age related diseases and disorders. Therefore, the current lipid and inflammation lowering invention provides a desirable preventive for reducing the risks for many of the age-related disorders. Treatment with the current invention should also lead to a significant drop in all-cause mortality.

There are many examples of prior art that have attempted to reduce inflammation with varying success. To reduce inflammation, the prior art has often focused on nonsteroidal anti-inflammatory drugs (NSAIDs) to inhibit the COX-1 and/or COX-2 enzymes. The NSAIDS are a family of medications used to treat mild-to-moderate pain or inflammation arising from many different disorders. However, NSAIDs have not been found to be beneficial in the prevention of the type of low-level chronic systemic inflammation that promotes cardiovascular disease with the possible exception of low-dose aspirin, which is in the prior art. After the FDA-approval of the COX-2-specific NSAIDs, many investigators hoped that these more specific NSAID drugs might help with chronic inflammation without the serious side effects of the nonspecific NSAIDs. While COX-2-specific NSAIDs did improve the gastrointestinal side effects of the non-specific NSAIDs, Merck's Vioxx apparently increases cardiovascular disease some 4-fold and has been withdrawn from the market, while new questions have been raised about the safety of Pfizer's COX-2-specific Cellebrex (the last remaining COX-2 specific NSAID that is still marketed in the US). Instead of using NSAIDs, the current invention uses pharmaceuticals and natural products (statins, alpha-lipoic acid, and corosolic acid) with demonstrated antiinflammatory activity and low toxicity.

The current invention specifies one further very important innovation not found in the prior art. C-reactive protein (CRP) is now considered a standard serum blood test for inflammation and a valuable index of future cardiovascular risk that is independent of cholesterol or glucose levels [22, 35, 44, 45]. While CRP tests are recommended by an increasing number of physicians as a guide to the patient's future cardiovascular risks and perhaps all-cause mortality, the CRP index suffers from the major drawback that there is currently no recognized pharmaceutical treatment that can be proscribed to lower high CRP values (typically >3 mg/dL) for all patients. Statins, alpha-lipoic acid and corosolic acid should synergistically lower CRP, along with the other inflammatory indicators IL-6 and TNF-alpha. Therefore, the current invention proposes a novel composition of statins in combination with alpha-lipoic acid and corosolic acid for lowering serum levels of CRP, IL-6 and TNF-alpha, which can be taken by those with high CRP levels (>3 mg/dL) but normal to high fasting cholesterol levels (>150 mg/dL) and normal to high glucose levels (>85 mg/dL). Using the current invention, testing for CRP levels may become as common as cholesterol tests are now, which would give an independent warning of cardiovascular risk and permit early intervention. By intervening very early in the degenerative process of cardiovascular disease to reduce high CRP levels, the current invention has the potential to reduce cardiovascular risks significantly lower than is currently possible using lipid reduction alone. Moreover, the said composition of statin, alpha-lipoic acid, and corosolic acid can be given as a small once-per-day tablet or capsule that lowers the risks of most age-related disorders and all-cause mortality for those over 40 years of age.

### SUMMARY OF THE INVENTION

Statin therapy has revolutionized the treatment of cardiovascular disease and is perhaps the major reason that cardiovascular disease rates have declined in the US over the last 30 years. Unfortunately, not all patients can take the required level of statins for optimally low cholesterol levels because of the side effects of statin drugs. Moreover, patients on statins for long periods apparently face increased risks of neuromuscular disease. Therefore, there is a need for effective cholesterol-lowering therapies with lower overall side effects than the solo statin drug therapies that currently dominate the prevention and treatment of cardiovascular disease. The present invention provides compositions that provide potent cholesterol lowering while minimizing the damaging side effects to liver, muscles, and neurons. The invention also provides highly desirable compositions and methods for reducing chronic systemic inflammation, which is an independent determinant of cardiovascular disease and all-cause mortality. The metrics for reduction of inflammation consists of lowering serum levels of high-sensitivity C-reactive protein (CRP), Interleukin 6 (IL-6), and/or tumor necrosis factor alpha (TNF-alpha).

Specifically, the current invention presents pharmaceutical compositions for reducing cholesterol and chronic systemic inflammation comprising therapeutically effective amounts of: at least one lipid-lowering agent chosen from HMG-CoA reductase inhibitors, niacin, red yeast rice, or policosanol; and at least one antiinflammatory natural product chosen from alpha-lipoic acid and corosolic acid. Typical pharmaceutical compositions to reduce high cholesterol with fewer drug side effects contain therapeutically effective amounts of simvastatin and alpha-lipoic acid. Typical pharmaceutical compositions to reduce moderate to high cholesterol and high inflammation contain therapeutically effective amounts of simvastatin, alpha-lipoic acid, and corosolic acid. To those in need of such treatment, the current invention also provides safe methods for reducing high cholesterol or chronic inflammation, or for reducing both high cholesterol and chronic inflammation simultaneously via treatment with therapeutically effective daily doses of said pharmaceutical compositions. The present invention provides subjects with compositions and methods for reducing cholesterol and inflammation as a prevention or treatment for many age-related diseases and disorders.

### DETAILED DESCRIPTION OF THE INVENTION

The present invention describes pharmaceutical compositions for reducing cholesterol and systemic inflammation comprising therapeutically effective amounts of: (Group A) at least one lipid-lowering agent chosen from HMG-CoA reductase inhibitors, high-dose controlled-release niacin, red yeast rice, or policosanol; and (Group B) at least one antiinflammatory natural product chosen from alpha-lipoic acid and corosolic acid. The pharmaceutical compositions of the present invention may optionally be formulated in single or multiple capsules, tablets, softgels, or liquid along with binder, emulsifying, fuller, stabilizing, sustained release, and/or carrier agents.

The said HMG-CoA reductase inhibitors within Group A are typically selected from the existing statin drugs: atorvastatin, cerivastatin, fluvastatin, lovastatin, mevastatin, pravastatin, rosuvastatin, and simvastatin. The term HMG-CoA reductase inhibitor (statin) is intended to include all pharmaceutically acceptable salt, ester and lactone forms of compounds which have HMG-CoA reductase inhibitory activity, and therefore the use of such salts, esters and lactone forms is included within the scope of this invention. The majority of the statins are produced by fermentation using microorganisms of different species belonging to Amycolatpsis, Aspergillus, Monascus, Mucor, Nocardia, or Penicillium genus. Some statins are the products of partial or total chemical synthesis.

Another possible Group A agent is policosanol, which is a recent Cuban alternative to the statins for lowering cholesterol. Policosanol is a plant derived long-chained aliphatic alcohol, which may be nearly as effective as the statins. Policosanol inhibits the biosynthesis of cholesterol in the liver by some mechanism that has yet to be elucidated. Policosanol has never undergone rigorous FDA trials and is therefore only available as an unapproved nutritional supplement of unknown reliability. Policosanol may be extracted from sugar cane or synthesized. The term policosanol includes all pharmaceutically acceptable salt, ester and lactone forms of long-chained aliphatic alcohols which have policosanol-like activity, and therefore the use of such salts, esters and lactone forms is included within the scope of the invention.

Other non-statin Group A agents include red yeast rice and high-dose controlled-release niacin. Red yeast rice (e.g. Monascus purpureus) is a nutritional supplement that contains a small amount of a statin-like compound, which could explain why it lowers cholesterol. However, the statin-like agent (lovastatin) in red yeast is found in very low concentrations compared to the pharmaceutically effective dose in statin drug therapy, so inhibition of HMG Co-A reductase may not be its major mechanism of action. This is also true of the lipid lowering agent niacin, where the lipid-lowering mechanism is not as well understood. Niacin must be taken in very high doses of 1 to 2 grams per day to be effective, which generates skin flushing and abdominal problems in most patients. Some form of controlled-release niacin is typically proscribed to avoid these side effects. While patients normally do not have the flushing or stomach problems with controlled-release niacin, liver toxicity and higher serum CRP level have been observed in some patients on extended-release niacin. The current invention that combines controlled-release niacin with alpha-lipoic acid and, optionally, corosolic acid should reduce the side effects of high-dose niacin therapy.

All of the above Group A agents are thought to lower serum cholesterol and low levels of serum cholesterol are highly correlated with reduced risks for all of the cardiovascular diseases. However, the HMG-CoA reductase inhibitors (statins) and the other lipid-lowering agents (policosanol, red yeast rice, and niacin) have side effects. Since policosanol and red yeast rice have only been tested in small clinical trials, their therapeutic effectiveness and full side effect profiles remain unknown. High-dose controlled-release niacin is well tested clinically and FDA approved, but the statin drugs are better at lowering LDL cholesterol and are therefore the preferred Group A agents. The HMG-CoA reductase inhibitors have been extensively tested in large clinical trials and are currently used by millions of patients throughout the world. The statin drugs are therefore the preferred lipid lowering Group A agents and improved statin safety is a major feature of the current invention.

Early warning of serious side effects of statin therapy (or other lipid-lowering agents) is often revealed in liver enzyme assays, as the liver is the main organ for metabolizing drugs and detoxifying the blood. If the liver is under stress from drug use, certain liver enzymes will be elevated, which usually leads to a recommendation to stop taking the drug. While liver inflammation is a more common warning sign of unwanted statin side effects, the most dangerous side effect is muscle inflammation, which can lead to serious kidney damage. Up to 10% of statin users may have a mild form of muscle toxicity with a feeling of tiredness. Statin drugs can destabilize mitochondria, leading to the release of cytochrome 450 and oxidative free radicals. In more extreme cases, mitochondria disintegrate and release iron groups into the general circulation that can lead to acute oxidative stress of the kidney and even kidney failure. As recognized by the current invention, alpha-lipoic acid acts on mitochondria to stabilize them against drug-induced damage. Therefore, drug-induced liver, muscle, and kidney inflammation from statins should be significantly reduced by combining alpha-lipoic acid with the statin HMG-CoA reductase inhibitors.

Long term use is the norm with statin therapy, but may present its own risks even if muscle or kidney damage does not appear during the first year of use. A large Danish study found that long-term statin users had a 4- to 14-times higher risk of developing peripheral neuropathy, which leads to weakness, pain, and trouble walking. Other studies have also shown an increased risk of peripheral neuropathy with longer term statin use. Some people who have been using atorvastatin for two years or more report symptoms similar to multiple sclerosis or ALS (Lou Gehrig's Disease). Both preclinical and clinical data has demonstrated that alpha-lipoic acid is an effective treatment for diabetic peripheral neuropathy and polyneuropathy (see background section above). As recognized by the current invention, alpha lipoic acid should significantly suppress the peripheral neuropathy and polyneuropathy that sometimes accompanies statin use.

Apart from the ability to mitigate the negative side effects of the Group A lipid-lowering agents, alpha-lipoic acid can also suppress LDL oxidation [46]. Oxidized LDL can not be degraded by normal cellular processes and thus produces inflammatory and other toxic effects in the endothelial cells forming the intima of the vessel wall. LDL oxidation is linked to many dysfunctions including increased platelet adhesion, increased levels of plasminogen activator inhibitor, decreased thrombomodulin, and changed levels of heparin sulfate proteoglycans. Oxidized LDL also activates inflammatory processes by up-regulation nuclear factor kappa-B, expression of adhesion molecules, and recruitment of monocytes/macrophages. This leads to higher levels of cytokines like Interleukin 6 (IL-6), tumor necrosis factor alpha (TNF-alpha), and high-sensitivity C-reactive protein (CRP). All of these dysfunctions are strongly associated with chronic inflammation and the subsequent development of atherosclerosis and other age-related disorders. Significantly, alpha-lipoic acid has also been shown to reduce pro-inflammatory serum levels of cytokines IL-1, IL-6, and TNF-alpha [27-30].

As recognized by the current invention, corosolic acid is also a potent antiinflammatory, as indicated by its ability to inhibit 12-O-tetradecanoylphorbol-13 acetate (TPA) induced inflammation with potency similar to the antiinflammatory drugs indomethasin and hydrocortisone [32, 33] and thus should counteract some or all of the inflammatory dysfunctions linked to atherosclerosis and the other age-related disorders. Given the current costs and commercial availability, the preferred source of corosolic acid is an alcohol-water extract of Lagerstroemia Speciosa L. standardized for corosolic acid. A Japanese 1% or 18% corosolic extract of Lagerstroemia Speciosa L is available from Softgel Technologies of Los Angeles. The 1% corosolic extract (GlucoTrim) has been used in weight loss products and also has potent glucose lowering activity [38]. However, unlike the glucose-lowering drugs rosiglitazone and pioglitazone, which also have independent antiinflammatory activity [34-36], corosolic acid does not significantly reduce glucose levels of non-diabetics [37] and thus may be safely used for its antiinflammatory properties in non-diabetics. Based on its apparent safety and antiinflammatory potency, the current invention recognizes that corosolic acid or plant extracts standardized for corosolic acid should be good agents for reducing chronic systemic inflammation.

Statins themselves also have significant antiinflammatory activity, as statins have reduced serum CRP, IL-6, and TNF-alpha in clinical trials [23-26]. This statin-induced reduction in inflammation is also linked to a decline in all-cause mortality [19, 23, 47]. As recognized by the current invention, therapeutically effective doses of statins are thus synergistic with Group B agents alpha-lipoic acid and corosolic acid as a method for lowering the early serum markers of inflammation (CRP, IL-6, and TNF-alpha). Clinical trials have shown that the said inflammatory markers (especially CRP and IL-6) are significant predictors of cardiovascular and neurodegenerative disease, which are independent of lipid status. Therefore, the statin plus alpha-lipoic acid plus corosolic acid compositions of the current invention can be taken in a once-per-day tablet or capsule, which comprise a useful treatment method for reducing said inflammatory risk markers as well as cardiovascular and neurodegenerative disease that is not currently available in the prior art.

As used herein, the therapeutically effective amount of a lipid-lowering Group A agent is the dose that reduces LDL or total cholesterol to a level that provides benefit in the prevention, treatment, and/or management of one or more conditions or diseases that are associated with high cholesterol and/or high lipid levels (namely the cardiovascular diseases). Typically, the goal is to reduce LDL cholesterol to below 100 mg/dL, or even better, to 70 to 80 mg/dL. The good HDL cholesterol should rise to above 40 mg/dL. The doses of lipid-lowering drugs needed to reduce LDL are well known in the prior art. For example, the HMG-CoA reductase inhibitor (or pharmaceutically acceptable salt thereof) atorvastatin (Lipitor) is provided as 10, 20, 40, or 80 mg tablets and most patients reach the desired LDL reduction with the lowest dose of one 10 mg tablet per day. Simvastatin (Zocor) is provided in 5, 10, 20, 40, and 80 mg tablets and most patients require one 20 mg tablet per day to reach acceptable LDL reduction. Likewise, the patient's cholesterol status and the prior art sets the desired dose for all the other HMG-CoA reductase inhibitors (cerivastatin, fluvastatin, lovastatin, mevastatin, pravastatin, and rosuvastatin). As examples, the typical daily dosage range varies for these alternative statins: cerivastatin has 0.1, 0.2, 0.3, 0.4, and 0.8 mg tablets; fluvastatin has 20, 40, and 80 mg tablets; lovastatin has 10, 20, 40, and 80 mg tablets; and pravastatin has 10, 20, and 40 mg tablets. Oral administration may be in single or divided doses of two to four times daily, although a single daily dose of the statin is the preferred regimen.

For high-dose niacin, the typical dose is 500 mg controlled-release tablets taken 2 to 4 times per day for a total of 1,000 to 2,000 mg of controlled-release niacin. For policosanol, the prior art uses a single 10 or 20 mg tablet per day, while red yeast rice requires at least two 600 mg capsules per day to have significant LDL reduction. The higher doses of niacin, policosanol, or red yeast rice will further reduce LDL, but can typically lead to more risks of side effects.

As used herein, the phrase therapeutically effective amount of anti-inflammatory alpha-lipoic acid is the dose that reduces low-level inflammation over a 4 to 6 month period as indicated by a significant reduction of serum levels of Interleukin 6 (IL-6), and/or tumor necrosis factor-alpha (TNF-alpha). Since alpha-lipoic acid is an antioxidant, care must be taken that the dose administered is high enough to reduce oxidative inflammation significantly, yet low enough to limit the inevitable pro-oxidation side effects found with high doses. Many published reports from short term clinical trials have used high 200 to 600 mg/day doses of alpha-lipoic acid, which appears optimal in short-term trials of 4 to 6 weeks. However, alpha-lipoic acid accumulates in body lipids over many months, so that human doses of 200 to 600 mg/day show a decline in therapeutic effectiveness over 2 to 5 months time frame. As recognized by the current invention, the therapeutic effective dose for humans of alpha-lipoic acid over the long term (4 months or more) likely ranges between 40 to 160 mg per day with the preferred intake likely to be in the range of 80 to 120 mg/day depending on the person's weight and other factors.

As used herein, the phrase therapeutically effective amount of anti-inflammatory corosolic acid is the dose that reduces low-level inflammation over a 4 to 6 month period as indicated by a significant reduction of serum levels of CRP, L-6, and/or TNF-alpha. Like alpha-lipoic acid, corosolic acid accumulates in body lipids over a period of months and is very active chemically. High accumulated doses of corosolic acid can lead to headaches or other side effects of low blood sugar. Thus, long term corosolic acid dosage should be limited to some 0.4 to 0.6 mg/day. When the corosolic acid source is a Lagerstroemia Speciosa L. extract standardized for 1% corosolic acid, some 40 to 60 mg/day of this plant extract in a lecithin or oil base (to assure bioavailability) is a predicted therapeutically effective dose range to reduce inflammation. If the corosolic acid source is an enriched Lagerstroemia Speciosa L. extract standardized for 18% corosolic acid, some 2.2 to 3.3 mg/day of the enriched plant extract in an oil base is predicted appropriate dose range.

The above dose ranges for alpha-lipoic acid and corosolic acid are calculated estimates based partly on solo treatment data in the absence of lipid lowering agents. Therefore, the optimal therapeutically effective doses for alpha-lipoic acid and corosolic acid are best determined in extensive 4 to 6 months human clinical trials along with a fixed amount of a statin like 10 mg/day atorvastatin (Lipitor) or 20 mg/day simvastatin. The final end points of the trial would be significant additional reductions in the serum levels of CRP, IL-6, and TNF-alpha over that found with statin alone.

Typical examples of Group A-B combination compositions of the current invention are given below in Table 1. Note that Table 1 does not contain complete formulations in that it lists only the pharmaceutically active lipid-lowering Group A agents and anti-inflammatory Group B natural agents and excludes the inert excipients or carriers of the pharmaceutical compositions. Since alpha-lipoic acid and corosolic acid both have low solubility in water, 50 to 200 mg of a carrier such as lecithin and/or oil such as rice blan or olive oil are typically added to the composition to increase bioavailability of these lipid-soluble agents. Note that Table 1 also does not list all possible or even all desirable combinations of Group A-B compositions. The Table 1 examples of Group A-B compositions are given solely for the purpose of illustration and are not to be construed as limitations of the present invention, as many variations of the present invention are possible without departing from the spirit and scope of the invention.

Table 1. Daily amounts of lipid-lowering agents from Group A plus anti-inflammatory natural products from Group B are shown. Compositions are named in the left lane. All HMG-CoA reductase inhibitor compositions start with CoA. All niacin compositions shown are controlled release. Red Yeast Rice is shortened to RYR1 in last row. Daily corosolic acid doses are shown in Table 1 as corosolic ext. (extracts), which are Lagerstroemia Speciosa L. extracts standardized for 1% corosolic acid. If Lagerstroemia Speciosa L. extracts standardized for 18% corosolic acid are used, then the therapeutically effective daily doses may drop to 2.2 to 3.3 mg/day range when appropriate lecithin or oil carriers are used. If purified corosolic acid is used with lecithin or vegetable oil carriers, then the therapeutically effective corosolic dose may drop to as low as 0.4 to 0.6 mg/day.

| Comp. | Lipid-Lowering | Antiinflammatory Natural Products |
|---|---|---|
| CoA1a | 10 mg atorvastatin | 90 mg alpha-lipoic acid |
| CoA1b | 10 mg atorvastatin | 90 mg alpha-lipoic acid + |
| | | 40 mg corosolic ext. |
| CoA1c | 40 mg atorvastatin | 100 mg alpha-lipoic acid. |
| CoA1d | 80 mg atorvastatin | 100 mg alpha-lipoic acid + |
| | | 60 mg corosolic ext. |
| CoA2a | 10 mg simvastatin | 90 mg alpha-lipoic acid + |
| | | 40 mg corosolic ext. |
| CoA2b | 20 mg simvastatin | 90 mg alpha-lipoic acid |
| CoA2c | 40 mg simvastatin | 90 mg alpha-lipoic acid + |
| | | 50 mg corosolic ext. |
| CoA3 | 0.2 mg cerivastatin | 100 mg alpha-lipoic acid |
| CoA4 | 40 mg fluvastatin | 90 mg alpha-lipoic acid + |
| | | 50 mg corosolic ext. |
| CoA5 | 40 mg lovastatin | 90 mg alpha-lipoic acid + |
| | | 50 mg corosolic ext. |
| CoA6 | 20 mg pravastatin | 90 mg alpha-lipoic acid + |
| | | 50 mg corosolic ext. |
| CoA7 | 40 mg mevastatin | 90 mg alpha-lipoic acid |
| CoA8 | 20 mg rosuvastatin | 90 mg alpha-lipoic acid |
| Policos | 10 mg policosinol | 90 mg alpha-lipoic acid |
| Niacin | 1500 mg niacin | 100 mg alpha-lipoic acid + |
| | | 40 mg corosolic ext. |
| RYR | 1200 mg RYR | 90 mg alpha-lipoic acid |

The Table 1 compositions are preferably formulated in a unit dosage form. The term “unit dosage form” refers to physically discrete units suitable as unitary dosages for human patients and other mammals with each unit containing a predetermined quantity of active material calculated to produce the desired therapeutic effect in association with suitable pharmaceutical carriers or excipients. In the case of the HMG-CoA reductase inhibitors, a single daily unit dose is preferable for most of the statin inhibitors. A single daily unit dose is also advisable for policosanol. In the case of high-dose niacin and red yeast rice compositions, the daily dose shown in Table 1 may be divided into 2 or 3 unit doses that are taken at different times throughout the day. The high-dose niacin of Table 1 is assumed to be a controlled release form, so as to minimize skin flushing or intestinal discomfort.

The compositions of Table 1 and the invention are typically administered in admixture with suitable pharmaceutical diluents, excipients, or carriers that vary depending on the format of the unit dosage form and consistent with prior art and conventional pharmaceutical practices. Since alpha-lipoic acid and corosolic acid both have low solubility in water, 50 to 200 mg of a carrier such as lecithin and/or oil such as rice bran or olive oil are typically added to the composition to increase bioavailability of the lipid-soluble agents.

The format of the unit dosage form varies dependent on whether oral tablets, capsules, softgels, or liquid units are used. For instance, for oral administration in the form of a tablet or capsule, the active drug composition of a Group A plus Group B can be combined with an oral, non-toxic, pharmaceutically acceptable, inert carrier such as lecithin, lactose, starch, sucrose, glucose, methyl cellulose, magnesium stearate, dicalcium phosphate, calcium sulfate, mannitol, sorbitol, sodium bicarbonate, and the like. Lecithin is the preferred carrier for increasing bioavailability. For oral administration in liquid form, the oral drug components can be combined with any oral, non-toxic, pharmaceutically acceptable inert carrier such as vegetable oil, lecithin, ethanol, glycerol, water, and the like. For oral administration as softgel, rice bran oil and lecithin are preferred carriers. Additionally, when desired or necessary, suitable binders, controlled-release agents, lubricants, disintegrating agents, and coloring agents can also be incorporated into the mixture. Suitable binders and controlled-release agents include starch, gelatin, natural sugars such as glucose or beta-lactose, corn sweeteners, natural and synthetic gums such as acacia, guar, konjack, or sodium alginate, carboxyl-methylcellulose, hydroxypropyl-methylcellulose, polyethylene glycol, waxes, and the like. Lubricants used in these dosage forms include sodium oleate, sodium stearate, magnesium stearate, micro-crystalline cellulose, sodium benzoate, sodium acetate, sodium chloride, silica, and the like. Disintegrators include, without limitation, starch, methylcellulose, agar, bentonite, xanthan gum, and the like. Other inactive components which can be incorporated into the compositions of the present invention include colorings, calcium carbonate, magnesium oxide, magnesium hydroxide, flavorings, preservatives, flow-enhancers, filling aids, essences, and other aesthetically pleasing components.

Examples of suitable forms for administration include pills, tablets, capsules, and softgels. The pill, tablet, capsule, or softgel can be coated with a substance capable of protecting the composition from disintegration in the esophagus but will allow disintegration of the composition in the stomach and mixing with food to pass into the patient's small intestine. The polymer can be administered alone or in combination with a pharmaceutically acceptable carrier, diluent or excipient substance, such as a solid, liquid or semi-solid material. Examples of suitable carriers, diluents and excipients include lecithin, vegetable oil, lactose, dextrose, sucrose, sorbitol, mannitol, starches, gum acacia, alginates, tragacanth, gelatin, calcium silicate, cellulose e.g., magnesium carbonate or a phospholipid with which the polymer can form a micelle.

In the present application, the term “pharmaceutically acceptable salts” shall mean non-toxic salts of the compounds employed in this invention which are generally prepared by reacting the free acid with a suitable organic or inorganic base. Examples of such salts include, but are not limited to benzoate, bicarbonate, sodium, calcium, acetate, laurate, malate, maleate, succinate, tannate, tartrate, benzenesulfonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrobromide, hydrochloride, hydroxynapthoate, iodide, isothionate, lactate, lactobionate, mandelate, mesylate, methylbromide, methylnitrate, methylsulfate, mucate, napsylate, nitrate, oleate, oxalate, pamoate, palmitate, panthothenate, phosphate/diphosphate, polygalacturonate, potassium, salicylate, stearate, subacetate, teoclate, tosylate, and valerate.

In practicing the Group A-B methods of the invention, combination therapy of Group A-B agents refers to administration of a first therapeutic effective amount of a lipid-lowering Group A agent and a second therapeutically effective amount(s) of at least one of the antiinflammatory Group B agents to prevent or treat cardiovascular and/or neurodegenerative disease or to reduce high serum levels of LDL cholesterol, CRP, IL-6, and/or TNF-alpha. Administration in combination therapy encompasses co-administration of the first and second amounts of the compositions of the combination therapy in a single simultaneous manner, such as in a single capsule or tablet having a fixed ratio of first and second amounts, or in multiple, separate capsules or tablets for each. In addition, such administration also encompasses use of each compound in a sequential manner.

The dosage regimen to prevent or treat cardiovascular and/or neurodegenerative disease or to reduce high serum levels of LDL cholesterol, CRP, IL-6 and/or TNF-alpha is selected in accordance with a variety of factors. These include the type, age, weight, sex, diet, genetics, and medical condition of the patient, the severity of the disease, the route of administration, pharmacological consideration such as the activity, efficacy, pharmacokinetics and toxicology profiles of the particular compound employed, whether a particular drug delivery system is utilized, and which drug combination treatment is chosen. Thus, the dosage regimen actually employed may vary widely and thus deviate from the examples of preferred dosage regimens set forth above in Table 1.

In other embodiments, the current invention also provides methods for reducing lipids and/or chronic inflammation in patients in need of such treatment with tablets or capsules containing the compositions of the invention as described by the above examples and the examples shown in Table 1. The methods for reducing lipids and/or chronic inflammation typically involve orally taking one of the compositions of the present invention once-per-day and monitoring cholesterol and inflammation at 6 and 12 months by clinically testing for cholesterol and inflammation markers. If a patient has high cholesterol (>200 mg/dL) but normal inflammation levels, then the current invention gives many options for safely treating the high cholesterol with a once-per-day lipid-lowering tablet containing a statin (e.g. atorvastatin) and alpha-lipoic acid (e.g. see CoA1a or CoA1c in Table 1). If the patient is suffering from high serum levels of cholesterol (>200 mg/dL) and has high inflammation as indicated by high serum levels of CRP, IL-6, and/or TNF-alpha, then the current invention provides the treatment option of a once-per-day Group A-B compositions containing a statin plus alpha-lipoic acid plus corosolic acid (e.g. see CoA1b and CoA1d in Table 1). If the patient's cholesterol is normal (150 to 200 mg/dL) but the patient has high inflammation levels (as indicated by high levels of CRP, IL-6, and/or TNF-alpha), then treatment can start with one of the weaker statin combination treatments with alpha-lipoic acid plus corosolic acid such as CoA2A in Table 2. Therefore, the invention provides several compositions and methods for treating chronic inflammation that can be used by patients with a wide variety of cholesterol levels, including those with cholesterol in the normal range.

The current invention also provides a method for following the antiinflammatory treatments to ensure that they are therapeutically effective: patients treated with any of the anti-inflammatory compositions like those in Table 1 can be tested every 6 to 12 months during the first year of use to determine if their serum levels of LDL cholesterol, CRP, IL-6, and/or TNF-alpha have been effectively controlled. Patients undergoing treatment with the compositions disclosed herein can be routinely monitored by measuring serum levels of total cholesterol, LDL cholesterol, HDL cholesterol, triglycerides, CRP, IL-6, and/or TNF-alpha by any of the clinical serum testing methods well known in the prior art. Periodic testing and analysis of the patient's blood samples permits modification of the treatment regimen during therapy so that optimal effective amounts of lipid-lowering Group A agents and antiinflammatory Group B agents are administered over the long term. In this way, the treatment regimen and dosing combinations of Group A and Group B agents is optimized to obtain therapeutic effectiveness for each individual patient in managing his or her specialized risks for cardiovascular and neurodegenerative diseases.

There are several potential advantages of the combination therapies disclosed herein. First, those patients who currently can not take statin drugs because of liver or muscle toxicity may now be able to do so using the methods and compositions of the current invention. Second, patients who currently use statin drugs, but cannot take the higher doses needed to lower their lipids to desired levels because of toxic side effects may now be able to take higher statin doses safely. Third, and most significantly, all patients, regardless of their lipid levels, should benefit from reducing low level chronic inflammation, as indicated by high serum levels of CRP, IL-6, and/or TNF-alpha. In summary, the combination therapies described herein have the potential to revolutionize the treatment of cardiovascular and neurodegenerative disease by administering a single tablet or capsule per day to patients that reduces the major serum factors known to generate higher risk for cardiovascular and neurodegenerative disease. The invention also provides a method for lowering chronic systemic inflammation that is relatively independent of a patient's cholesterol level.

The terms ‘patient’ include any mammal, who take a lipid altering or antiinflammatory agent for any of the cardiovascular or neurodegenerative uses described herein. Administration of lipid-lowering Group A agents and/or anti-inflammatory Group B agents includes both self-administration and administration to the patient by another person. Humans are the preferred ‘patients’ for the compositions and methods described herein. However, the term ‘patient’ also includes non-human mammals such as dogs, cats, and horses.

While many embodiments of the invention have been disclosed above and include presently preferred embodiments, many other embodiments and variations are possible within the scope of the present invention. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention.

What is claimed is:

1. Pharmaceutical compositions for reducing cholesterol and chronic systemic inflammation comprising therapeutically effective amounts of: (A) at least one lipid-lowering agent chosen from HMG-CoA reductase inhibitors, high-dose controlled-release niacin, red yeast rice, or policosanol; and (B) at least one antiinflammatory natural product chosen from alpha-lipoic acid and corosolic acid.

2. The pharmaceutical compositions of claim 1 wherein said lipid lowering agent is a therapeutically effective lipid-lowering dose of a HMG-CoA reductase inhibitor drug such as atorvastatin, cerivastatin, fluvastatin, lovastatin, mevastatin, pravastatin, rosuvastatin, and simvastatin, and the pharmaceutically acceptable salts, esters, lactones and isomeric forms thereof.

3. The pharmaceutical compositions of claim 1 wherein said lipid lowering agent is a therapeutically effective lipid-lowering dose of a nutritional supplement chosen from high-dose controlled-release niacin at 0.5 to 2.0 grams per day, red yeast rice (e.g. Monascus purpureus) at 0.6 to 1.8 grams per day, and policosanol at 5 to 30 mg per day.

4. The pharmaceutical compositions of claim 1 wherein said alpha-lipoic acid contains a therapeutically effective antiinflammatory dose of synthetic or natural alpha-lipoic acid or any of the alpha-lipoic acid analogs and the pharmaceutically acceptable salts, esters, lactones and isomeric forms thereof.

5. The pharmaceutical compositions of claim 1 wherein said corosolic acid contains a therapeutically effective antiinflammatory dose of purified synthetic or natural corosolic acid, or said corosolic acid is provided by ethanol-water extracts of plants such as Lagerstroemia speciosa or Perilla frutescens, whereby said plant extracts are standardized for one percent by weight corosolic acid or higher, and said corosolic acid also contains the pharmaceutically acceptable salts, esters, lactones and isomeric forms thereof.

6. Pharmaceutical compositions of claim 1 wherein said lipid lowering agent is 10, 20, 40, or 80 mg/day atorvastatin and said antiinflammatory natural product is 80 to 120 mg/day alpha-lipoic acid.

7. Pharmaceutical compositions of claim 1 wherein said lipid lowering agent is 10, 20, 40, or 80 mg/day atorvastatin and said antiinflammatory natural products include both 80 to 120 mg/day alpha-lipoic acid and 0.4 to 0.6 mg/day corosolic acid, whereby said corosolic acid can alternatively be provided by 40 to 60 mg/day of Lagerstroemia Speciosa L. extracts standardized for 1% by weight corosolic acid, or by a comparable dose of said Lagerstroemia Speciosa L. extracts standardized for 18% by weight corosolic acid.

8. Pharmaceutical compositions of claim 1 wherein said lipid lowering agent is 5, 10, 20, 40, or 80 mg/day simvastatin and said antiinflammatory natural product is 80 to 120 mg/day alpha-lipoic acid.

9. Pharmaceutical compositions of claim 1 wherein said lipid lowering agent is 5, 10, 20, 40, or 80 mg/day simvastatin and said antiinflammatory natural products include both 80 to 120 mg/day alpha-lipoic acid and 0.4 to 0.6 mg/day corosolic acid, whereby said corosolic acid can alternatively be provided by 40 to 60 mg/day of Lagerstroemia Speciosa L. extracts standardized for 1% by weight corosolic acid, or by a comparable dose of said Lagerstroemia Speciosa L. extracts standardized for 18% by weight corosolic acid.

10. Pharmaceutical compositions of claim 1 wherein said lipid lowering agent is 10, 20, or 40 mg/day pravastatin or lovastatin and said antiinflammatory natural product is 80 to 120 mg/day alpha-lipoic acid.

11. Pharmaceutical compositions of claim 1 wherein said lipid lowering agent is 10, 20, or 40 mg/day pravastatin or lovastatin and said antiinflammatory natural products include both 80 to 120 mg/day alpha-lipoic acid and 0.4 to 0.6 mg/day corosolic acid, whereby said corosolic acid can alternatively be provided by 40 to 60 mg/day of Lagerstroemia Speciosa L. extracts standardized for 1% by weight corosolic acid, or by a comparable dose of said Lagerstroemia Speciosa L. extracts standardized for 18% by weight corosolic acid.

12. Pharmaceutical compositions of claim 1 wherein said lipid lowering agent is 0.1, 0.2, 0.3, or 0.4 mg/day cerivastatin and said antiinflammatory natural product is 80 to 120 mg/day alpha-lipoic acid.

13. Pharmaceutical compositions of claim 1 wherein said lipid lowering agent is 20, 40, or 80 mg/day fluvastatin or lovastatin and said antiinflammatory natural product is 80 to 120 mg/day alpha-lipoic acid.

14. Pharmaceutical compositions of claim 1 wherein said lipid lowering agent is 500, 1000, 1500, or 2000 mg/day controlled-release niacin and said antiinflammatory natural product is 80 to 120 mg/day alpha-lipoic acid.

15. Pharmaceutical compositions of claim 1 wherein said lipid lowering agent is 500, 1000, 1500, or 2000 mg/day controlled-release niacin and said antiinflammatory natural products include both 80 to 120 mg/day alpha-lipoic acid and 0.4 to 0.6 mg/day corosolic acid, whereby said corosolic acid can alternatively be provided by 40 to 60 mg/day of Lagerstroemia Speciosa L. extracts standardized for 1% by weight corosolic acid, or by a comparable dose of said Lagerstroemia Speciosa L. extracts standardized for 18% by weight corosolic acid.

16. Pharmaceutical compositions of claim 1 wherein said lipid lowering agent is 600, 1200, or 1800 mg/day red yeast rice and said antiinflammatory natural product is 80 to 120 mg/day alpha-lipoic acid.

17. Pharmaceutical compositions of claim 1 wherein said lipid lowering agent is 5, 10, 20, or 30 mg/day policosinol and said antiinflammatory natural product is 80 to 120 mg/day alpha-lipoic acid.

18. The compositions of claims 1 wherein each composition is formulated in single or multiple capsules, tablets, softgels, or liquid along with binder, emulsifying, fuller, stabilizing, controlled release, and/or carrier agents such as: polyethylene glycol, magnesium stearate, magnesium oxide, magnesium hydroxide, calcium carbonate, lecithin, vegetable oils, silicone dioxide, starch, stearic acid, microcrystalline cellulose, gelatin, carboxyl-methylcellulose, and hydroxypropyl-methylcellulose.

19. The methods for reducing cholesterol and chronic systemic inflammation comprising the oral administration of therapeutically effective daily doses of any of the compositions of claim 1 to mammals in need of such treatment.

20. The methods of claim 19 wherein the 4 to 6 month markers of success in reducing cholesterol and chronic systemic inflammation comprise reductions in the serum levels of LDL cholesterol, high-sensitivity C-reactive protein, interleukin 6, and/or tumor necrosis factor alpha.
